(19) United States
(12) Patent Application Publication
(10) Pub. No.: US 2019/0159947 A1
(43) Pub. Date: May 30, 2019

(54) DIAPER SIZE EXTENDER AND METHOD OF USE

(71) Applicant: Denisse C. Mercado, Fort Worth, TX (US)

(72) Inventor: Denisse C. Mercado, Fort Worth, TX (US)

(21) Appl. No.: 16/205,966

(22) Filed: Nov. 30, 2018

Related U.S. Application Data
(60) Provisional application No. 62/592,788, filed on Nov. 30, 2017.

(51) Int. Cl. A61F 13/58; A61F 13/49; A61F 13/56

(52) U.S. Cl. CPC A61F 13/581; A61F 13/622; A61F 13/5638; A61F 13/49011

(57) ABSTRACT

A diaper tab to be secured at a side opening of a diaper, the diaper tab having an elongated body; an adhesive tab extending from a first side of the elongated body; and a hook and loop fastener extending from a second side of the elongated body; the first side is opposite the second side; the adhesive tab is to secure to a first side of the side opening and the hook and loop fastener is to secure to a second side of the side opening; and the elongated body is positioned within the opening.

### BACKGROUND

### 1. Field of the Invention

The present invention relates generally to diapers, and more specifically, to a tab for extending the size of a diaper for extended use of a single diaper size.

### 2. Description of Related Art

Diapers are well known in the art and are used for infants and children before potty training. For example, FIG. 1 depicts a conventional diaper 101 having two tabs 103, 105 for securing the diaper to a child. During use, the diaper is fitted to the child and closed therearound via the two tabs.

One of the problems commonly associated with diaper 101 is improper fit. For example, it is common for a child to outgrow a conventional diaper size before the parents run out of the particular size. Therefore, the parents typically throw away the unused diapers. In addition, even a diaper of a correct size may not fit a child properly.

Accordingly, although great strides have been made in the area of diapers, many shortcomings remain.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIG. 1 is an oblique view of a common diaper;

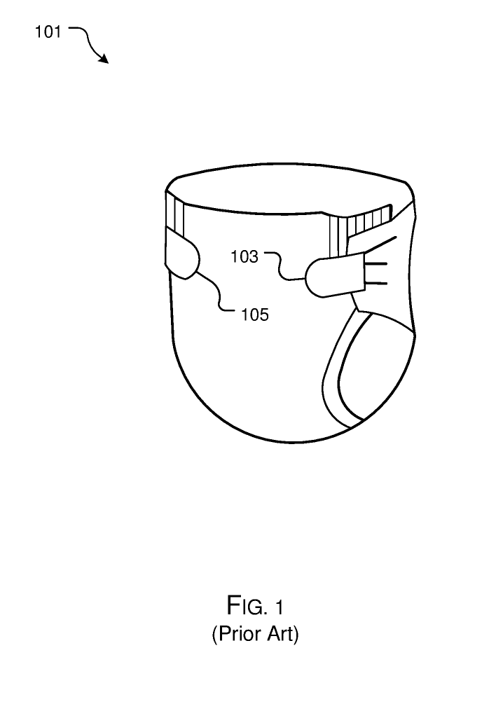

FIG. 2 is an oblique view of a diaper system in accordance with a preferred embodiment of the present application;

FIG. 3 is a tab for a diaper from FIG. 2; and

FIG. 4 is a flowchart of the method of use of the system of FIG. 2.

While the system and method of use of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the system and method of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The system and method of use in accordance with the present application overcomes one or more of the above-discussed problems commonly associated with conventional diapers. Specifically, the present invention provides a means to increase the size of a diaper and improve the fit of a diaper. These and other unique features of the system and method of use are discussed below and illustrated in the accompanying drawings.

The system and method of use will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless described otherwise.

The preferred embodiment herein described is not intended to be exhaustive or to limit the invention to the precise form disclosed. It is chosen and described to explain the principles of the invention and its application and practical use to enable others skilled in the art to follow its teachings.

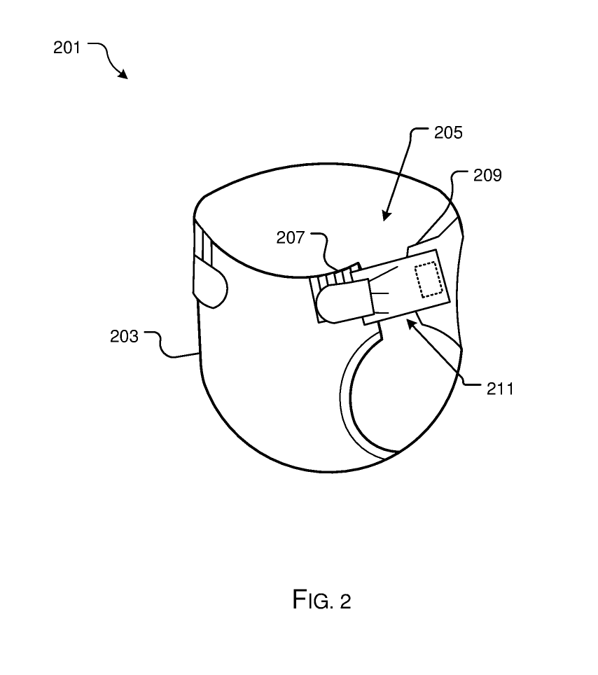

Referring now to the drawings wherein like reference characters identify corresponding or similar elements throughout the several views, FIG. 2 depicts an oblique view of a diaper system in accordance with a preferred embodiment of the present application. It will be appreciated that system 201 overcomes one or more of the above-listed problems commonly associated with conventional diapers.

In the contemplated embodiment, system 201 includes a conventional diaper 203 with an opening 205 between a first side 207 and a second side 209. It should be appreciated that the size and stile of conventional diaper 203 can vary. System 201 further includes a diaper tab 211 configured to secure to and extend from first side 209 to second side 207, thereby extending the size of diaper 203.

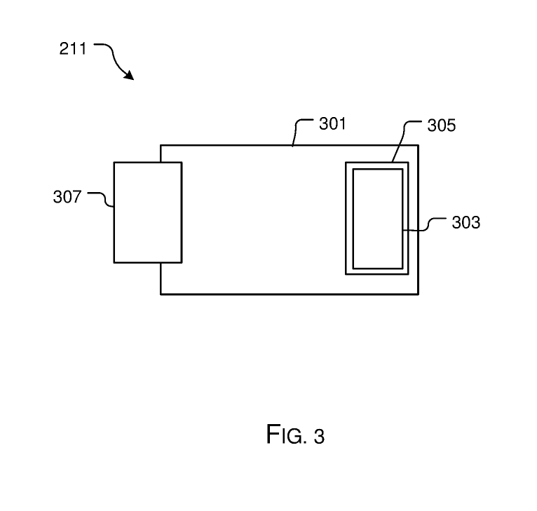

In FIG. 3 a top view of tab 211 is shown. Tab 211 includes an elongated body 301 having an adhesive 303 at a first side. It is contemplated that the adhesive 303 can have a covering 305 to be peeled off before securing to a side of diaper 203. In the preferred embodiment, adhesive 303 is non-toxic, thereby being safe for being used near a child's skin. In addition, in the preferred embodiment, elongated body 301 is composed of a soft, elastic material, such as a cotton/spandex blend, thereby allowing for body 301 to stretch. It should be appreciated that the overall size, materials, color, and shape of body 301 can vary as aesthetical, functional, or manufacturing considerations require.

Tab 211 further includes a second tab 307 extending from a second side of elongated body 301. In the preferred embodiment, tab 307 is a hook and loop fastener configured to secure to the body of diaper 203.

It should be appreciated that one of the unique features believed characteristic of the present application is the combination of the adhesive, elongated body, and second tab, configured to extend the size of a diaper, thereby allowing for a more comfortable fit of the diaper.

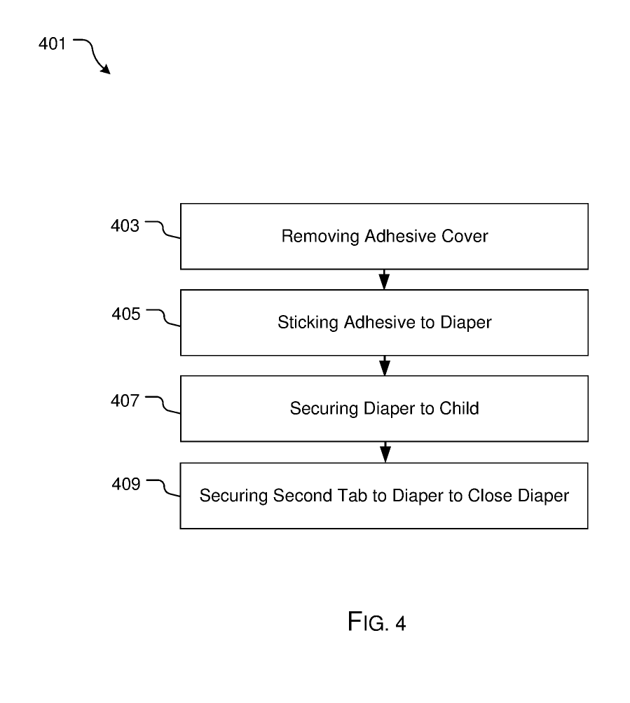

In FIG. 4, a flowchart 401 depicts a method of use of tab 201. During use, the adhesive cover is removed so that the adhesive can secure to a side of the diaper, as shown with boxes 403 and 405. The diaper is secured to the child by placing the diaper around the child's body and securing the second tab to the side of the diaper opposite the adhesive, as shown with boxes 407, 409.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed is:

1. A diaper tab configured to be secured at a side opening of a diaper, the diaper tab having:
an elongated body;
an adhesive tab extending from a first side of the elongated body; and
a hook and loop fastener extending from a second side of the elongated body;
wherein the first side is opposite the second side;
wherein the adhesive tab is configured to secure to a first side of the side opening and the hook and loop fastener is configured to secure to a second side of the side opening; and
wherein the elongated body is positioned within the opening.

2. The tab of claim 1, wherein the elongated body is rectangular.

3. The tab of claim 1, wherein the elongated body is composed of an elastic material.

4. The tab of claim 1, further comprising:
a removable covering of the adhesive tab.
